(19) United States
(12) Patent Application Publication
(10) Pub. No.: US 2007/0231879 A1
(43) Pub. Date: Oct. 4, 2007

(54) Perfusion device for the treatment and examination of an object in a liquid

(76) Inventors: Steffen Hering, Wien (AT); Sascha Hering, Innsbruck (AT)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/393,226

(22) Filed: Mar. 30, 2006

(51) Int. Cl. C12M 1/34

(52) U.S. Cl. 435/287.1; 435/293.1

(57) ABSTRACT

A perfusion device (10) is described, particularly for electro-physiological and/or optical examinations on biological objects (1) which comprises a chamber body (20) in which a perfusion chamber (21, 25) is formed with at least one input opening (22, 23) and at least one discharge (24), and a holding device (30) for holding at least one object (1) to be examined in the perfusion chamber, the holding device (30) comprising a projection that forms a seat (31) on its upper end for the object (1) to be examined and is level-adjustably arranged in the perfusion chamber (21, 25) and, if the holding device (30) protrudes into the perfusion chamber (21, 25), a circumferential perfusion gap (25) being formed laterally and relative to the chamber body (20), which gap is connected to the discharge (24).

### FIELD OF THE INVENTION

The invention concerns devices for object treatment and object examination in a liquid bath, particularly perfusion devices with a perfusion chamber for biological cells, and methods for the application of the devices.

### BACKGROUND OF THE INVENTION

Open or closed (covered off) perfusion chambers are known from the literature, in which measurements can be carried out with measurement electrodes or patch-pipettes. Where open systems are concerned, tube-shaped perfusion chambers are used where a liquid (treatment solution) is put in through a tube connection and removed again through a second tube connection. The speed of the solution change depends on the solution flow and on the chamber volume. An acceleration of the perfusion can be achieved by means of a reduction of the chamber volume. For closed perfusion chambers, a cover plate is provided on the perfusion chamber. Measurement electrodes can be inserted into the perfusion chamber through openings in a chamber wall or in the cover plate (refer, for example, to DE 43 05 405 C1).

Perfusion devices are also known, which are designed specially for a quick perfusion of isolated and larger objects such as xenopus oocytes. These cells are to be examined simultaneously, for example, with two microelectrodes. With reference thereto, S. Hering in “Pflüger Archive”, Vol. 436, 1998, P. 303-307, describes a perfusion chamber that is covered off with a glass plate. Measurement electrodes can be inserted through openings in the glass plate in such a way that the cell can be penetrated by the electrodes and the process can be observed through the glass plate. One opening in the glass plate is surrounded by a funnel. A filling of the solution into the funnel and the subsequent application of a low pressure at the chamber discharge enable the perfusion of the chamber and the objects (e.g. xenopus oocytes) contained therein. With a multiple application of solutions into the funnel, it is endeavored to suction off liquid volumes from the chamber, which are equal in size in each individual case.

The construction as described by S. Hering can have disadvantages with regard to the following aspects. In the first instance, the opening with the second measurement electrode surrounding the funnel is only closed off by way of the liquid layer between the electrode and the inner rim of the opening. Therefore, if the low pressure in the chamber exceeds the force of the closing liquid meniscus, air can enter the chamber through this second opening and this can lead to damage to the cell and, subsequently, to disturbances in the measurement sequence. A further disadvantage of the potential air inlet lies in the fact that an automatic operation is made difficult. For example, with a continual operation of the perfusion chamber, equal solution volumes are to be injected into the funnel and suctioned off at the discharge. This would only be possible with frequent corrections as a result of the air inlet. With the repeated application of a strong low pressure to the chamber, air enters practically inevitably through the second opening. Subsequently, and with equal off-suction volume, correspondingly less liquid is removed and a disturbing residual volume is formed in the supply funnel. An automated operation, where equal volumes are to be repeatedly put in and drawn off, is not possible with this chamber construction.

Secondly, a quick exchange of the perfusion solution is desirable for many examinations on objects such as, for example, xenopus oocytes. This concerns in particular measurements for the characterization of receptor-activated ion channels. A slow supply frequently leads to desensitization of the receptors even during the application of the agonist and, subsequently, to incorrect measurement results. With the chamber construction as described by S. Hering et al., the solution is applied laterally to the cell lying on the bottom of the chamber. The abrupt liquid flow leads to a “hammer” effect, meaning, the cell is hit from the side by a pressure impulse. Depending on the speed and duration of the liquid flow, this lateral pressure impulse can lead to translocations of the oocytes and, ultimately, to a flushing of the cells out of the chamber. In this case the contact of the cell with the electrodes is disturbed initially and later interrupted, a situation that leads to the termination of the measurement. The contact of measurement electrodes with the cell is, however, very sensitive with regard to mechanical disturbances. Even minor mechanical disturbances lead to changes in the sealing of the electrodes at the oocyte membrane. For this reason, a lateral pressure impulse is very disadvantageous for stable and reproducible measurements. Therefore, the use of the conventional device is restricted to experimental work and is not suitable for routine or automated measurements.

A further disadvantage of the technique as described by S. Hering involves a residual volume occurring in particular as a result of capillary forces and adhesion of residual liquid between the electrodes and the inner wall of the funnel. With the capillary forces, supply and removal of the liquid to and from the funnel can not be adequately synchronized. Moreover, a mixing of the liquids in the funnel can occur, subsequently leading to a thinning of the concentration of test substances.

### OBJECTIVE OF THE INVENTION

The objective of the invention is to provide an improved perfusion device with which the disadvantages of the conventional perfusion devices are overcome and which enables in particular a treatment and/or examination of biological objects such as, for example, biological cells with increased stability and reliability. Furthermore, the improved perfusion device should enable in particular the operation with increased perfusion rates and a continual, automated operation. It is also the objective of the invention to provide improved methods for the examination and/or treatment of biological objects such as, in particular, biological cells in perfusion devices where, in particular, work can be performed with reduced liquid volumes while maintaining a high selectivity at the same time. The invention should enable a complete and reliable exchange of the liquid in the volume of the perfusion chamber of a perfusion device, particularly with minor liquid volumes in the range of, e.g., 50 to 200 μl. In this case, the balance between supplied and outgoing liquid volumes is to be ensured, also for a sequence of numerous measurement cycles.

### SUMMARY OF THE INVENTION

With reference to the device, the above objective is solved according to the invention with a perfusion device having a perfusion chamber formed in a chamber body and a holding device arranged in the perfusion chamber for the object to be examined to that extent that the holding device has a projection, which can be positioned protruding into the perfusion chamber in such a way that a liquid flowing to a discharge of the perfusion chamber flows around the projection laterally on all sides. The holding device comprises a preferably cylindrical-shaped part that is level-adjustably incorporated in the chamber bottom and that is encompassed by a (perfusion-) gap, which is connected to the discharge. The perfusion chamber forms the circumferential gap (annular gap, perfusion gap) around the holding device relative to the chamber body. The discharge can be provided in the chamber body or in the holding device. The perfusion gap preferably has a circular ring-shaped cross-section. The resulting, preferably coaxial arrangement of the holding device with the circumferential perfusion gap has the advantage that, with the passage of a liquid through the perfusion chamber, there is an all-round flow about the seat of the holding device and subsequently of the object to be examined, so that the one-sided pressure impulse occurring in a conventional perfusion chamber is avoided. The solution flows around the object from all sides into the preferably coaxial perfusion gap and is drawn off either into a side channel (discharge) or, as an alternative, through openings in the holding device. Both technical solutions conduct an approximately laminar flow around the cell into the perfusion gap. The object that comprises, for example, a single biological cell, a cell group or a compound of cell constituents, is circumflowed from the at least one input opening into the perfusion chamber towards the discharge in the laminar flow.

According to a preferred embodiment of the invention, the holding device is adjustably arranged in the perfusion chamber in such a way that the position of the seat for the object to be examined in the perfusion chamber and particularly along the length of the perfusion gap is changeable. This embodiment can have advantages with reference to the adaptation of the chamber geometry to the object to be examined.

According to a further advantageous embodiment of the invention, the perfusion device according to the invention is provided with an electrode holder for positioning at least one measurement electrode. One or several measurement electrodes are used particularly for electro-physiological examinations on biological cells. With the electrode holder, the measurement electrodes can be positioned permanently fixed relative to the perfusion chamber and, therefore, relative to the object to be examined. During examination procedures, the measurement electrodes themselves also have a mechanical holding function for the object.

According to a particularly preferred embodiment of the invention, the perfusion device is provided with a cover plate which closes off the perfusion chamber in the upward direction and in which the at least one input opening is formed into the perfusion chamber. The use of the cover plate that is preferably separable from the chamber body of the perfusion device can have advantages with regard to a simplified access to the chamber interior, for example for placing down an object for examination on the holding device or for cleaning purposes. Furthermore, the cover plate forms an evaporation protection. A liquid loss resulting from evaporation is reduced. The reproducibility of the control during liquid supply and removal can be increased.

If, according to one variant, an input funnel is provided that is connected to the at least one input opening, advantages can result with regard to the liquid supply into the perfusion chamber. According to a configuration of the invention, the input funnel is a part of the chamber body, and this fact can have advantages for a simplified and integral structural arrangement of the perfusion device. However, it is preferred if the input funnel is arranged as part of the above-mentioned cover plate on its upper side so that, by the cover plate, a bottom of the input funnel is formed in which the at least one input opening is formed and under which the perfusion chamber in assembled condition is located. Then, for loading or cleaning the perfusion chamber, the structure comprising cover plate and input funnel can be removed advantageously and in a particularly easy manner from the perfusion device.

There are particular advantages for the perfusion device according to the invention if the cover plate has two input openings, which are formed on the bottom of the input funnel and arranged in the assembled condition of the cover plate with the chamber body symmetrically relative to the longitudinal direction of the projection formed by the holding device in the perfusion chamber. This variant of the invention has, first of all, the advantage that the tub-shaped funnel includes both input openings for liquid supply and/or for access by the measurement electrodes. In this way, the disadvantageous entry of air into the perfusion chamber, as described above for the conventional perfusion devices, is avoided in any case. The liquid can enter through both input openings in the same way, and this excludes the access of air. There is a second advantage in the symmetrical arrangement of the input openings above the seat for the object to be examined. In this way, the symmetrical circumflow of the object from all sides from the input openings to the coaxial perfusion gap is improved. By means of the symmetrical circumflow of the cells from all sides into the coaxial perfusion gap, lateral forces do practically not occur. Mechanical disturbances of the measurements or object treatments are avoided as a result.

Finally, there is a further advantage of the integration of the input openings in the bottom of the input funnel in the fact that, due to the size of the input openings (according to the circumstances, reduced by the deployed measurement electrodes), the flow of a liquid filled into the input funnel is delayed into the perfusion chamber. If a new liquid, e.g., a test solution, is filled into the input funnel on the cover plate, there is at first merely a diffusion from the funnel through the remaining intermediate space between the measurement electrodes and the internal sides of the input openings into the perfusion chamber. Only after the application of a low pressure at the discharge of the perfusion chamber will the liquid be drawn through the input openings symmetrically in the direction of the object to be examined. The low pressure can be set in such a way that this flow movement is considerably faster than the diffusion movement. This advantageously enables a solution change in the perfusion chamber with a sharply segregated and relatively quickly moving liquid front (limitation between various treatment, rinsing or test solutions). The timing control of the object treatment or the object examination can be carried out with high level accuracy and precision.

If both input openings according to a preferred configuration have the same diameter, the symmetrical exertion of the flow forces is advantageously improved to a further degree.

The cover plate with the input funnel is advantageously arranged laterally displaceable on the chamber body. In an advantageous manner, the perfusion chamber can subsequently be selectively opened or closed without having to separate the cover plate with the input funnel from the chamber body completely.

According to a further advantageous embodiment of the invention, the holding device in the perfusion chamber has the form of a cylinder. This configuration improves the uniformity of the flow through the perfusion gap up to the discharge.

If the holding device according to a further embodiment of the invention has on its upper end a holder of the deepening for centrally receiving the object under examination, further advantages can result for the stabilization of the object in the liquid flow.

According to a further variant, a screw connection for fixation in the chamber body can be provided on the lower end of the holding device, through which the adjustment capability of the holding device as described above is advantageously facilitated. An important advantage of the invention lies in the fact that the object to be examined can be level-adjustably placed in the perfusion chamber by means of the variable (e.g., by means of a threaded connection) connection of the cylinder-shaped projection (perfusion platform). This variable level-adjusting capability has the following advantages: firstly, the measurement electrodes laterally inserted through the cover plate into the chamber can be adjusted with regard to their angle. The adjusting capability of the injection angle is advantageous. This change can be corrected by the adaptation of the height of the perfusion platform, formed by the holding device, in the perfusion chamber. This is particularly advantageous for examinations on objects of various sizes where the injection into the cell must take place in the exact lateral position and at a certain angle. Secondly, the level-adjusting capability of the holding device enables the selective positioning of the cell, either higher or deeper in the perfusion gap. This has effects on the time between applying a low pressure at the perfusion channel and the cell perfusion, but also on the stability of the cell in the liquid flow during the measurement process. A deeper lowering of the cell in the perfusion chamber by lowering the holding device prolongs the time until the solution in the funnel reaches the cell and stabilizes the cell in the laminar flow as the effect of lateral (according to the circumstances, asymmetrical) forces onto the surface of the cell in deeper sections of the perfusion channel is further reduced.

With a further embodiment of the invention, it is provided that the holding device consists at least partially of a transparent material, which enables the illumination and/or observation of the objects under examination from the direction of the bottom side of the chamber body. In particular, a light guide can be placed in the holding device, which light guide has an immediate contact with the object under examination at its light exit surface. In this way, for example, light can be supplied to the cell during measurement and/or light signals such as fluorescence signals can be taken from the surface.

If the holding device according to a further variant of the invention has a reference electrode, measurement errors during electrical measurements on the object under examination can be avoided or diminished.

Further advantageous embodiments of the invention are characterized in that the discharge from the perfusion chamber is connected to a pump and/or a reference electrode is arranged in the discharge. Both features serve an improved usage of the perfusion device according to the invention for automated operation.

If the perfusion device according to the invention is provided with at least one auxiliary tube, further advantages in terms of liquid level adjustment in the perfusion chamber, washing the perfusion chamber and protecting the perfusion chamber against unintended drying are obtained.

A further subject-matter of the invention is an examination apparatus for biological objects, particularly biological cells, cell groups or cell constituents, which comprises a dosage device, a measurement device and the perfusion device according to the invention. The dosage device serves the particular purpose of the automatic supply of various liquids (particularly treatment, rinsing and test solutions) from storage reservoirs into the perfusion chamber. The measurement device is set up, in a manner known as such, for electro-physiological and/or optical measurements on objects treated in the perfusion chamber.

With reference to the method, the above-mentioned objective is solved by means of a method for the examination and/or treatment of at least one biological cell in the perfusion device according to the invention with the following steps. After a positioning of the at least one cell on the holding device, a pre-determined volume of a treatment solution is filled into the sample chamber and a measurement is performed on the at least one cell. By applying a low pressure to the discharge of the perfusion chamber or the funnel discharge of the input funnel, the treatment solution can be removed. This procedure has the particular advantage that the positioning of the at least one cell on the holding device is stabilized by the liquid flow through the perfusion chamber and the reliability of the measurement is increased accordingly.

According to advantageous variants of the invention, a fluorescence measurement and/or an electro-physiological measurement are provided as a measurement on the cell, through which the reaction of the cell to the treatment solution and/or electric stimulus can be advantageously characterized.

The treatment solution can be advantageously put in with a dosage device into the sample chamber that is operated time-synchronous with the control of the removal of the liquid. Here, various test reservoirs are preferably used in the dosage device, from which the individually desired treatment solution is filled into the sample chamber, particularly into the input funnel of the sample chamber. The directions of the liquid supply and removal can be reversed by filling in the treatment solution through the discharge into the sample chamber and removing it through the funnel discharge. In this case, advantages can result for a more flexible application of the perfusion device.

If, according to a variant of the invention, partial volumes are suctioned off through the discharge and the funnel discharge, the sum of the partial volumes being equal to the filled-in volume of the treatment solution, advantages can result for the liquid dosage. In particular, a larger solution volume than is required for the perfusion can be supplied at first into the input funnel with a low-level dosage accuracy, and then a surplus is removed by way of the funnel discharge so that the liquid volume in the sample chamber can be adjustably set with a high degree of accuracy.

According to one variant, there can advantageously be a continual supply of the treatment solution with a simultaneously continual discharge from the perfusion chamber. As an alternative, a discontinuous operation can be advantageous for certain measuring methods.

### BRIEF DESCRIPTION OF THE DRAWINGS

Further details and advantages of the invention are explained in the following description with reference to the attached drawings. The drawings show in:

FIG. 1: a schematic and enlarged cross-sectional view of a first embodiment of the perfusion device according to the invention;

FIG. 2: a schematic top view onto a perfusion device according to FIG. 1;

FIG. 3: a schematic and enlarged cross-sectional view of a further embodiment of the perfusion device according to the invention; and

FIG. 4: a schematic illustration of an examination apparatus according to the invention that is equipped with the perfusion device according to the invention.

### DETAILED DESCRIPTION OF THE INVENTION

The invention is described as follows in an exemplary manner with reference to an embodiment where the perfusion device is provided with a cover plate with integrated input funnel. It is emphasized here that the implementation of the invention is not limited to this configuration but is an alternative to that extent that no cover plate or a cover plate without an input funnel or an integral structural arrangement of the chamber body with the input funnel is provided. It is furthermore emphasized that the illustrations shown here are not scale drawings. The absolute sizes and size relationships and conditions can be selected depending on the concrete application requirements when implementing the invention.

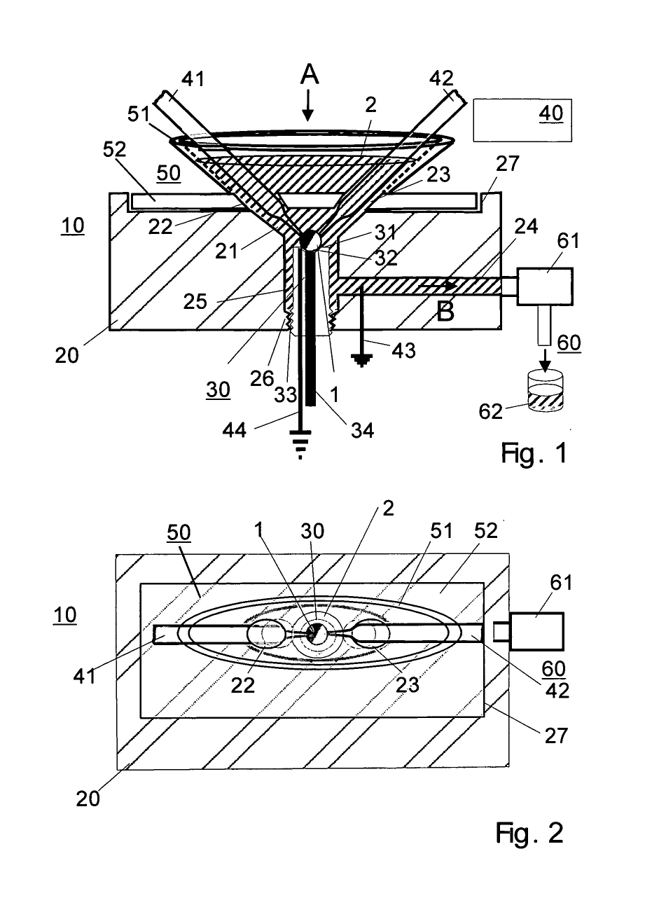

The embodiment of a perfusion device 10 according to the invention, as shown in the FIGS. 1 and 2 in schematic cross-sectional view and in top view, respectively, comprises the chamber body 20, the holding device 30, the electrode holder 40, the cover plate 50 and the collecting device 60. Further details, known as such, of the perfusion device such as a holder in a laboratory equipment unit or connecting parts for connecting to measurement, additional or control equipment/devices are not shown in the FIGS. 1 and 2 for reasons of clarity.

The chamber body 20 is formed by a plastic part in which the sample chamber 21, 25 and the discharge 24 are integrated. The chamber body 20 consists, for example, of PTFE or of another suitable synthetic material. The perfusion chamber 21, 25 comprises an upper chamber zone 21, through which the incoming liquid flows towards the object to be treated, and a lower chamber zone 25 which, with an introduced holding device 30, essentially forms the annular or perfusion gap, through which the liquid flows to the discharge 24. The discharge 24 is connected to the collecting device 60, which is equipped with a pump 61 and a collecting reservoir 62.

The diameter of the cell 1 is normally smaller than 1 mm, e.g., for oocytes about 800 μm, or even smaller than 500 μm. The volume of the sample chamber 21, 25 is, for example, about 10 to 15 μl. This means that, with the supply of e.g. 50 to 100 μl of a treatment solution, the inner volume of the sample chamber is substituted several times. The diameter of the discharge 24 is, for example, 500 μm to 5 mm.

On the lower side the chamber body 20 is provided with a threaded bore 26, into which the holding device 30 can be screwed (see below). On the upper side of the chamber body 20, a recess 27 is provided whose inner extension is adapted to the size of the cover plate 50 (see below).

The holding device 30 is formed by a cylinder-shaped projection whose upper end forms a seat 31 for the object to be examined (cell 1) and whose lower end is provided with an external thread 33 for engaging into the threaded bore 26. The cylinder-shaped projection extends in the vertical direction upwards into the sample chamber 21, 25. At the seat 31, a hemispherical recess 32 is provided, which stabilizes the cell 1 in the examining position. At least one light guide 34 for optical or measurement purposes is integrated in the holding device 31, which light guide 34 is arranged between the seat 31 at the upper end of the holding device 30 and a measuring device (not shown).

The merely schematically shown electrode holder 40 serves the purpose of a permanently fixed positioning of the measurement electrodes 41, 42 relative to the perfusion device 10. The electrode holder 40 comprises for example a frame known as such, possibly with a 3D-precision setting device. As an alternative, the measurement electrodes 41, 42 are attached to manipulators, which are standing on holders, which are mechanically joined to the chamber body 20. The measurement electrodes 41, 42 are pushed through the input openings 22, 23 into the sample chamber.

The perfusion device 10 is provided with at least one of the following reference electrodes. A first reference electrode 43 is arranged in the discharge 24. It can be advantageous for ion current measurements if two reference electrodes are used. For this purpose, a second reference electrode 44 ought to be in the immediate vicinity of the cell, at which the measurements are carried out. In this way, influences of interference resistances (e.g., series resistances) on the measurement are minimized.

In an advantageous embodiment, one of the reference electrodes 44 is integrated in the holding device 30, the electrode protruding from the holder in the immediate vicinity of the cell. In this way, the reference potential can be formed immediately beside the cell. As an integral component of the holding device, the electrode 44 can be detached by means of the threaded union 33 from the chamber body and easily replaced. This is advantageous in particular with the use of, for example, silver-silver chloride electrodes, which have to be renewed and/or exchanged after a certain amount of time.

In accordance with this independent aspect of the invention, the holding device 30 can be provided as an electrode assembly or one-way electrode for screwing into a perfusion device 10.

The cover plate 50 is essentially a plane component made of a transparent material such as, for example, of glass or synthetic material. The cover plate 50 consists of an input funnel 51, which is arranged on the base part 52 with the input openings 22, 23. The base part is a rectangular plate that is fitted into the recess 27 of the chamber body 20. As an alternative, the recess 27 can form just a one-sided or a two-sided stop for the cover plate 50 and, moreover, can enable a displacement of the cover plate 50 on the chamber body 20, so that the upper chamber zone 21 of the perfusion chamber can be released. The input openings 22, 23 run corresponding to the desired alignment of the measurement electrodes inclined with a pre-determined angle relative to the plate plane through the cover plate 50.

In order to carry out the method according to the invention, the perfusion chamber is first filled with a rinsing liquid, such as, for example, a physiological salt solution. After this, at least one cell 1 to be examined is positioned on the holding device 30. For this purpose, the cover plate 50 is taken off or shifted so that the perfusion chamber 21, 25 is exposed. The cell 1 is placed, for example with a Pasteur pipette, onto the seat 31. Then the measurement electrodes 41, 42, depending on the desired examination method, are positioned in a manner known as such in such a way that the measurement electrodes touch the cell 1 or penetrate it. After this, a treatment liquid 2 is filled into the input funnel 51 (see arrow A in FIG. 1). At first, the treatment solution represents a liquid front relative to the rinsing liquid existing in the perfusion chamber 21, 25. By activating the pump 61, the rinsing liquid is drawn off (see arrow B in FIG. 1), so that the treatment solution flows in from the input funnel 51 into the perfusion chamber 21, 25. Depending on the desired measurement protocol, the desired optical and/or electrical measurement on the cell 1 takes place either at the same time or with a certain delay. Then, and as required, further treatment solutions can be supplied by way of the input openings 22, 23 into the perfusion chamber 21, 25.

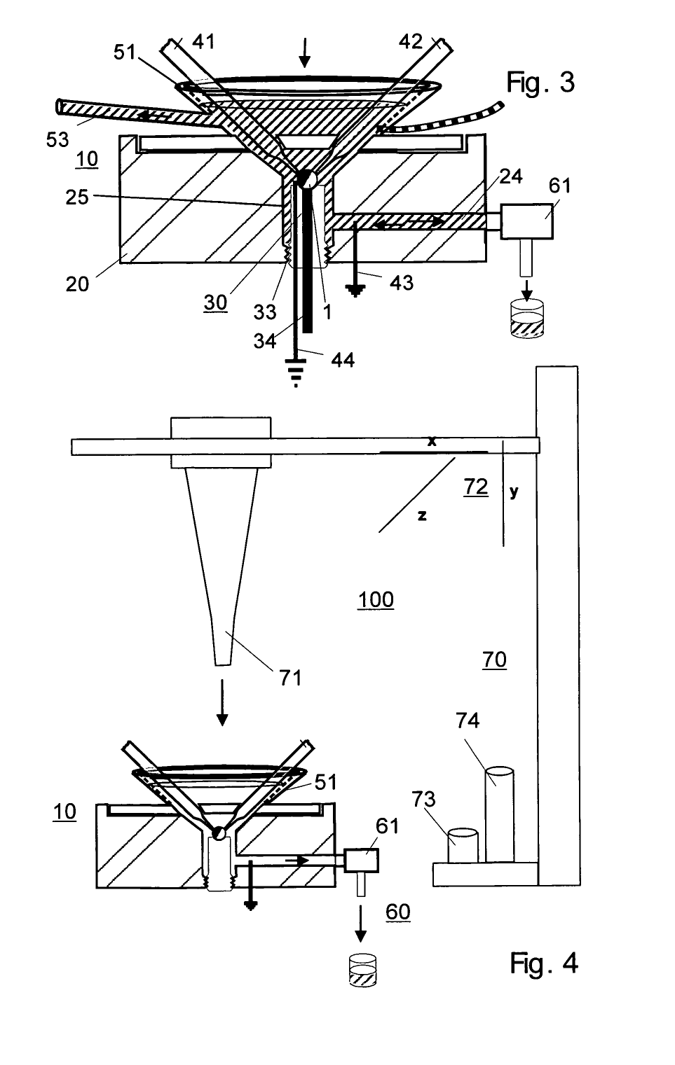

With the embodiment of the invention as shown in FIG. 3, at least one lateral funnel discharge 53 is provided additionally at the input funnel 51, which discharge is connected to a suction pump (not shown). This funnel discharge 53 is arranged preferably above the base part 51 in a section of the funnel wall, which is located in the immediate vicinity to the position of one or both electrodes 41, 42. The diameter of the funnel discharge 53 in the funnel wall is, for example, 500 μm to 2 mm.

By applying a low pressure to the funnel discharge 53, a residual volume can be removed quickly and reliably from the sample chamber 21. A mixing of residual liquid and newly applied liquid in the input funnel 51 is thereby advantageously avoided. In a process-technical sense, it is ensured that the residual volumes are quickly removed and, even in the event of a supply of a solution surplus, this can be corrected quickly.

The positioning of the at least one additional funnel discharge 53 into the funnel wall has the further advantage that, if required, liquid can be supplied into the sample chamber 21 also from below by way of the discharge 24 and can be continually suctioned off through the funnel 51. By means of a solution supply to the discharge 24 and continual suctioning of the solution through the funnel discharge 53, the sample chamber (between the quick solution change e.g. with the application of an agonist into the funnel) can be continually (from below in the upward direction) perfused. With this usage, the application spectrum of the chamber is considerably extended and the stability of the measurements is increased.

Particularly with regard to the embodiment of the invention with the funnel discharge 53, the base part 52 of the cover plate 50 in the interior of the funnel 51 can have at least one additional free opening into which no microelectrode is inserted. In this case, the solution can selectively come from the funnel into the interior of the sample chamber or, in reverse, can flow from the chamber into the funnel. Free openings that are located beside the electrode openings can be useful for reasons of symmetry in particular or for pressure equalization.

According to a further embodiment of the invention, the funnel 51 can be provided with at least one auxiliary tube, which is essentially arranged like the funnel discharge 53 (see dotted line). Preferably, two auxiliary tubes are positioned at side walls of the funnel 51 in opposite relationship to each other. A particular advantage of the auxiliary tubes is obtained, when the volume supplied to the funnel 51 is larger (or smaller) than the volume withdrawn through the discharge 24. Accordingly, the adjustment of the liquid level in the perfusion chamber can be improved. Furthermore, by using the auxiliary tubes, any complete drying of the chamber can be avoided. Finally, liquids can be removed from funnel corners with difficult access.

For carrying out the method according to the invention it can be provided that, at first, less test solution is suctioned off from the funnel than was filled in with the pipette device and the residual volume remaining in the funnel is removed from the funnel by way of an additional suction pump via the funnel discharge 53 in the funnel wall.

A complete examination apparatus according to the invention, which is suitable for the method sequence as described, is shown schematically in FIG. 4. The examination apparatus 100 comprises the dosage device 70 and the measurement device (not shown) in addition to the perfusion device 10 according to the invention. The dosage device 70 comprises a liquid conveying device 71 (e.g., pipette, syringe or similar), which is movable with a 3D-actuator 72 between liquid reservoirs 73, 74 and the input funnel 51. The liquid conveying device 71 is connected to a control device (not shown), with which the pump 61 of the collecting device is also connected, in order to enable a time-synchronous operation of the pump 61 and the liquid conveying device 71.

A further application of the method according to the invention lies in the fact that, between the discrete applications of test solutions into the input funnel as described above and the suctioning of the solution through the discharge 24, this discharge 24 can also be selectively used for the supply of solutions. In this case, the solution exits through the openings in the glass plate into the funnel and is removed through the funnel discharge 53. With the continual perfusion of the oocytes between the discrete applications, for example, the washing-out of test substances from the oocyte membrane is improved. After stopping the continual solution supply by way of discharge 24, the residual volume is removed from the funnel by way of the funnel discharge 53 and, selectively, the supply of discrete solution volumes into the input funnel with the pipette device can be continued.

The features of the invention disclosed in the above description, the drawings and the claims can be of significance both individually as well as in combination for the realization of the invention it its various embodiments.

We claim:

1. A perfusion device, for electrophysiological or optical examinations on biological objects, comprising:
a chamber body in which a perfusion chamber is formed with at least one input opening and at least one discharge, and
a holding device for holding at least one object to be examined in the perfusion chamber, wherein
the holding device comprises a projection that forms a seat on its upper end for the object to be examined and is level-adjustably arranged in the perfusion chamber, where, if the holding device protrudes into the perfusion chamber, a circumferential perfusion gap is formed laterally and relative to the chamber body, which gap is connected to the discharge.

2. The perfusion device according to claim 1, further comprising an electrode holder for the positioning of at least one measurement electrode, which electrode holder protrudes into the perfusion chamber.

3. The perfusion device according to claim 1, further comprising a cover plate, with which the perfusion chamber is covered off in the upward direction and in which at least one input opening is formed.

4. The perfusion device according to claim 1, further comprising an input funnel, which is connected to at least one input opening.

5. The perfusion device according to claim 4, wherein the input funnel is a part of the chamber body.

6. The perfusion device according to claim 4, wherein the input funnel is a part of the cover plate.

7. The perfusion device according to claim 4, wherein the input funnel has at least one funnel discharge, through which the liquid can be removed from the input funnel.

8. The perfusion device according to claim 4, wherein the cover plate has two input openings, which are formed on the bottom of the input funnel and are arranged symmetrically relative to the longitudinal direction of the holding device.

9. The perfusion device according to claim 8, wherein the two input openings have the same diameter.

10. The perfusion device according to claim 3, wherein the cover plate is arranged laterally displaceable, so that the perfusion chamber can be selectively opened or closed.

11. The perfusion device according to claim 1, wherein the holding device has the form of a cylinder.

12. The perfusion device according to claim 11, wherein the cylinder at the upper end has a half-round recess for centrally receiving the object.

13. The perfusion device according to claim 1, wherein the holding device at its lower end is connected to the chamber body by means of a threaded connection.

14. The perfusion device according to claim 1, wherein the holding device is at least partially a transparent material.

15. The perfusion device according to claim 1, wherein the holding device is provided with a light guide.

16. The perfusion device according to claim 1, wherein the holding device has a reference electrode.

17. The perfusion device according to claim 1, wherein the discharge is connected to a pump.

18. The perfusion device according to claim 1, wherein a reference electrode is arranged in the discharge.

19. The perfusion device according to claim 1, wherein at least one auxiliary tube is connected with the perfusion chamber.

20. An examination apparatus for biological objects, comprising:
a dosage device,
a measurement device, and
the perfusion device according to claim 1.

21. A method for the examination of at least one biological object in a perfusion device according to claim 1, comprising the steps of:
positioning the at least one object on the holding device,
filling in a defined volume of a treatment solution into the perfusion chamber,
conducting a measurement on the at least one object,
applying a low pressure to the discharge, and
withdrawing the treatment solution through the perfusion gap into the discharge or through the funnel discharge.

22. The method according to claim 21, wherein the measurement comprises at least one of a fluorescence measurement and an electro-physiological measurement.

23. The method according to claim 21, wherein the treatment solution is filled in with a dosage device and, corresponding to a predetermined time control, a volume is suctioned off through at least one of the discharge and a funnel discharge which suctioned volume is equal to the filled-in volume of the treatment solution.

24. The method according to claim 23, wherein partial volumes are suctioned off through the discharge and the funnel discharge.

25. The method according to claim 23, wherein, with the dosage device, the treatment solution is taken up from various test reservoirs and filled in through a nozzle into the sample chamber.

26. The method according to claim 23, wherein the treatment solution is filled in through the discharge into the sample chamber and the volume is suctioned off through the funnel discharge, which volume is equal to the filled-in volume of the treatment solution.

27. The method according to claim 21, wherein the treatment solution is continually filled into the perfusion chamber and is withdrawn at the same speed continually through the discharge or the funnel discharge while the measurement is being carried out on the at least one object.

28. The method according to claim 21, wherein the treatment solution is filled discontinuously into the perfusion chamber and, correspondingly, is withdrawn discontinuously through the discharge or the funnel discharge, the measurement on the at least one object being performed while the treatment solution is in a steady state.

29. The method according to claim 21, wherein a liquid level in the perfusion chamber is adjusted by introducing or withdrawing the treatment solution or a washing solution through at least one auxiliary tube.

30. A method for the examination of a biological cell, comprising the step of:
measuring a membrane potential of the cell.

31. The method according to claim 30, wherein the membrane potential of the cell is measured with the patch-clamp technique.
